(19) United States
(12) Patent Application Publication
(10) Pub. No.: US 2002/0122815 A1
(43) Pub. Date: Sep. 5, 2002

(54) Compositions and methods of carbohydrate dosing

(76) Inventor: Stephen J. Peroutka, Burlingame, CA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/947,766

(22) Filed: Sep. 7, 2001

Related U.S. Application Data
(60) Provisional application No. 60/259,569, filed on Jan. 4, 2001.

(51) Int. Cl. A61K 47/00; A61K 31/70

(52) U.S. Cl. 424/439; 514/23

(57) ABSTRACT

The present invention relates to compositions and methods for dosing the brain and body with adequate and steady glucose supplies, and functional foods to achieve these effects. Carbohydrate dosing can be used, for example, to maintain optimal serum glucose levels, to provide steady energy to the brain, to manage weight, and to treat and prevent chronic conditions. The present invention also relates to modulating the activation of the sympathetic nervous system, for treating and preventing disorders and conditions associated with disturbances of the sympathetic nervous system, such as migraine, headaches, and associated conditions, for treating and/or preventing headaches, and for treating and/or preventing migraines and/or associated symptoms.

This application claims the benefit of U.S. Provisional Application No. 60/259,569, filed Jan. 4, 2001, which is hereby incorporated by reference in its entirety.

### BACKGROUND OF THE INVENTION

Although eating is necessary to maintain the biological processes of every organism, it has profound adverse effects on an organism's health and homeostasis.

### DESCRIPTION OF THE DRAWINGS

FIG. 1 summarizes the body's physiological response to ingestion of a single meal containing carbohydrates.

FIG. 2 compares the effect of a low and high GI food on serum glucose levels after ingestion.

### DESCRIPTION OF THE PRESENT INVENTION

The present invention relates to functional foods and drugs, and methods of their administration, that provide controlled doses of glucose to meet an organism's energy requirements. These compositions supply adequate amounts of glucose to the brain, while minimizing, e.g., caloric intake, insulin release, serum glucose fluctuation, and/or sympathetic nervous system activation. As a result, many of the adverse consequences associated with improper carbohydrate dosing can be prevented and treated.

Glucose and other carbohydrates are the most powerful ingested “drugs” that affect brain function, yet they are rarely “dosed” properly in the modem diet. The body's response to serum glucose levels can be particularly disruptive, having adverse effects on a diverse number of physiological phenotypes, including, e.g., headache, migraine attacks, body weight, stress, memory, concentration, premenstrual syndrome (PMS), depression, chronic pain and other neuropsychiatric disorders. The amounts of total carbohydrates and sugars that are commonly listed on food labels have little or no significance in how they affect serum glucose levels, making it particularly difficult to determine appropriate carbohydrate dosages to avoid or minimize disruptive physiological events. The present invention provides methods and compositions for treating and preventing the adverse consequences of improper carbohydrate dosing.

The Biology of Eating

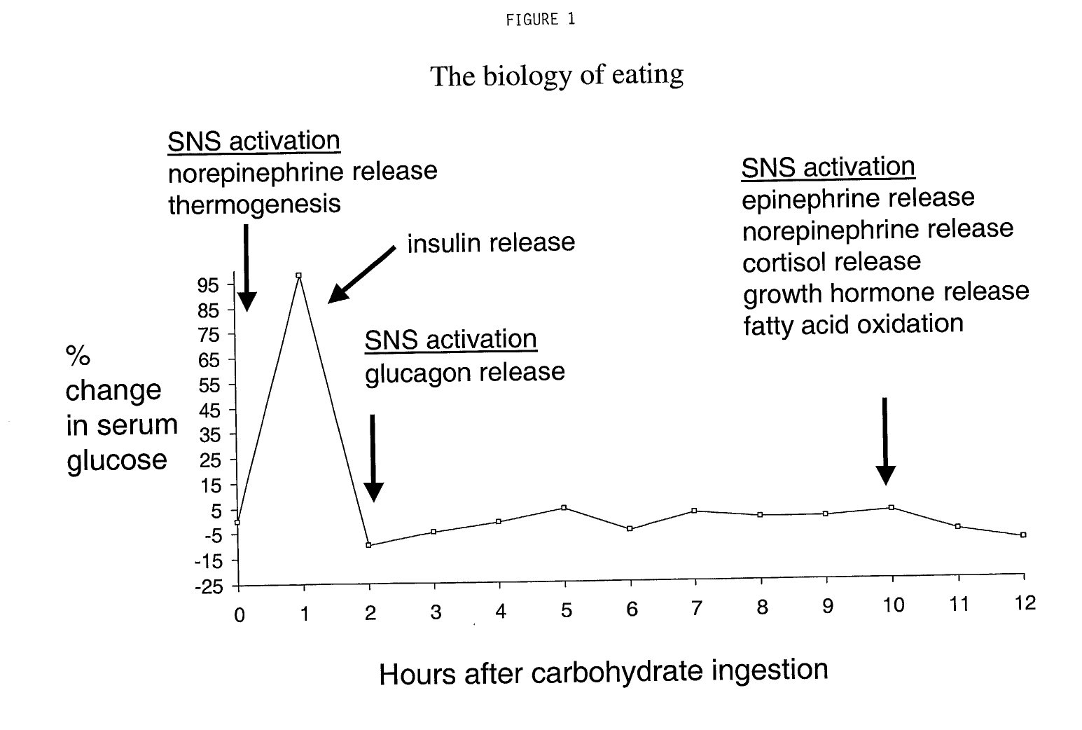

The body's physiological response to ingestion of a single meal containing carbohydrates is shown in FIG. 1. Many of the events that occur during this period are a result of the body's attempt to maintain the serum glucose at levels adequate to meet the energy requirements of the brain and body. One of the central players in this process is the sympathetic nervous system (SNS), a part of the autonomic nervous system that reacts to changes in the internal and external environment. The SNS is involved in a wide variety of physiological processes, one of which is the response to changes in serum glucose levels. Glucose levels fluctuate most commonly as a result of the absorption of carbohydrates, accounting for approximately 50% of the calories in typical diets in the U.S. and Europe.

Immediately after carbohydrate ingestion, serum glucose levels rapidly increase, stimulating the SNS and the associated release of the neurotransmitter norepinephrine. This is followed by a decline in glucose levels upon activation of the insulin pathway. Insulin levels induced by glucose changes typically occur in the blood after about 1-2 minutes, stimulating the uptake of glucose into the cells. These rapid changes in serum glucose levels (both in the positive and negative direction) activate the SNS, producing changes in cranial vasculature, cerebral vasodilation, cerebral blood flow, etc., responsible for some of the adverse effects of improper carbohydrate intake. As the serum glucose levels fall, the SNS is activated again, and the hormone glucagon is released. Glucagon acts as the physiological counterbalance to insulin and acts to increase serum glucose concentrations.

After about 2-12 hours of fasting, depending largely upon the amount of glycogen stores in the liver, the SNS is again stimulated, resulting in the release of epinephrine, norepinephrine, cortisol, growth hormone, and the stimulation of fatty acid oxidation, in efforts maintain steady serum glucose levels. Glucose levels are then maintained via the process of gluconeogenesis, in which glucose is created de novo from fat and protein precursors.

One of the objects of the present invention is to maintain substantially stable serum glucose levels, thereby avoiding or minimizing the body's physiological responses stimulated when such levels fluctuate. While not wishing to be bound to any mechanism, it is proposed herein that efforts by the body to maintain steady serum glucose levels, rather than the absolute levels of glucose, can be responsible for many of the short-term and long-term consequences of inadequate carbohydrate dosing, such as headache, migraine attacks, stress, and anxiety. Moreover, controlling serum glucose levels can have pronounced beneficial effects on efforts to maintain body weight, stress, memory, concentration, PMS, depression, chronic pain, and other neuropsychiatric disorders.

By modulating the rate of change in serum glucose levels, it has been found, in accordance with the present invention, that these the conditions can be treated and/or prevented. These changes can occur either acutely (e.g., after eating a meal), or after prolonged fasting when glucose levels are maintained largely as a result of gluconeogenesis. However, it is also an object of the present invention to treat these conditions, such as headache and migraine attack, with carbohydrate dosing and modulation, irrespective of the mechanism(s) that produce it.

Glycemic Index

The amounts and type of carbohydrate ingested have a direct effect on serum glucose levels. As mentioned, the food-labeling scheme currently in use for sugars and carbohydrates does not provide complete or correlative information on how their ingestion effects serum glucose levels. Instead, it has been recently been discovered that the glycemic index (GI) is more predictive of the outcome on serum glucose levels of ingesting a carbohydrate-containing food. As a result, the present invention uses the glycemic index as selection factor to achieve optimal carbohydrate dosing. This is not to say, however, that other indices would not work as well.

The glycemic index (“GI”) is a measure of the immediate changes in serum glucose levels that occur after the ingestion of carbohydrates. The GI is defined as the incremental area under the serum glucose response curve that results from a 50 g carbohydrate ingestion, as a percentage of the results obtained with an ingestion of a standard meal (usually 50 g glucose). Depending on the carbohydrate ingested, serum glucose can rise to varying degrees in the course of the next hour or two. Carbohydrates that are rapidly digested and absorbed have a high GI. The higher the GI of a food, the more rapid and significant will be the serum glucose changes, in both the positive and negative directions. By contrast, foods with a low GI (e.g., peanuts=14; yogurt=33) will cause a relatively slow and small rise in serum glucose, even though they may contain the same amount of carbohydrates as a food with a high GI (e.g. glucose=100; pretzels=83). For the purposes herein, a high GI food has a GI index of about greater than 75. Examples include, but are not limited to, glucose, donuts, waffles, breakfast bars, corn flakes, pretzels, potatoes, and rice. A low GI food, having a GI index of about less than 50, includes but are not limited to, fructose, oatmeal, yogurt, milk, many fruits, pasta, sponge cake, and Peanut M&Ms.

The GI refers to the physiological effects of carbohydrates. Importantly, the GI is unrelated to the chemical composition of the food. Indeed, the amount of “Total Carbohydrates” and “Sugars”, values that are listed on Food Labels, are now recognized as having no physiological significance. Glucose and fructose are both “simple” monosaccharides or sugars, yet they have significantly different GI values (100 and 23, respectively). By choosing a food with a low GI, an individual can significantly reduce their serum glucose fluctuations, insulin secretion, and sympathetic nervous system activation in comparison to eating foods with the same amount of carbohydrates, but having a high GI.

All carbohydrates, even more complex carbohydrates like starches or dextrins, are eventually metabolized into simple sugars (i.e., glucose, fructose or galactose). The end product of carbohydrate metabolism is always the same (i.e., glucose) in terms of the energy needs of the nervous system. The major difference between carbohydrates is that some food sources, like starches in breads, are 100% available for immediate conversion into glucose. By contrast, other sources of carbohydrate, like fruits, are only 50% available for immediate conversion into glucose. Another key fact is that the GI values for the monosaccharides vary tremendously. Fructose, for example, has one of the lowest GI values of any food (GI=23). Therefore, some complex carbohydrates have a higher “glycemic” effect on serum glucose levels than simple sugars.

A number of factors are known to affect the GI. These factors are summarized below.

Type of Sugar

The chemical structure of the sugar has a major effect on its metabolism. The frequently cited statement that “refined sugar” is not as advantageous as “natural sugars” is simply false. Many foods with large amounts of refined sugar have a GI value near 60. This is due to the fact that sucrose, or common table sugar, is a chemical combination of glucose (GI=100) and fructose (GI=23). Although both glucose and fructose are monosaccharides, they vary greatly in their GI values. Therefore, the GI of sucrose (60-65) is the average GI value of its two monosaccharide constituents, glucose and fructose.

Fructose is a monosaccharide that is a natural component of many fruits and honey. All pure, commercially available fructose comes from starch, usually cornstarch. Fructose is almost twice as sweet as sucrose, so that fewer calories need to be ingested to obtain the same level of sweetness. It has been estimated that the average American ingests about 37 g of fructose per day, an amount that represents approximately 8% of the daily carbohydrate energy needs.

Fructose is much more slowly absorbed from the gastrointestinal system than is glucose and many other carbohydrates. Moreover, fructose itself has no direct energy value. Usually, fructose is first converted in the liver to glycogen, which is then converted to glucose, as needed. As a result, fructose ingestion causes a relatively slow rise and fall in serum glucose levels. In fact, fructose has one of the lowest GIs indexes of any food (GI=23). Therefore, the use of fructose is an excellent means to minimize serum glucose fluctuations while maintaining the liver supply of glycogen. For instance, functional foods utilized for bedtime snacks can comprise effective amounts of carbohydrate-containing foods that have fructose to build liver glycogen pools, and avoid gluconeogenesis. Maintenance of an adequate supply of liver glycogen is the primary goal of carbohydrate dosing so that the physiological stress required for gluconeogenesis can be avoided.

Mannitol and sorbitol are naturally occurring sugar alcohols that cause less of a rise in serum glucose than glucose or sucrose. In large amounts, however, they may cause diarrhea.

Finally, complex carbohydrates also vary greatly in terms of their GI. Once again, the commonly held opinion that refined sugar raises serum glucose more than most complex starchy foods like bread, is simply false. Some starches, such as amylopectin, are much more rapidly metabolized than amylose starches. Amylose is a long, straight chain of sugars that are stored compactly and, as a result, more difficult to digest than highly branched starches like amylopectin. Thus, the GI of pretzels (GI=83) is much higher than table sugar (GI=60-65). The GI can therefore be decreased by increasing the ratio of amylose to amylopectin in starchy foods.

As a result, the GI value of food is cannot be predicted from the Food Label listing of total carbohydrates and sugars. The GI value can only be estimated if the exact carbohydrate constituents of a food are known. Even then, however, a number of other food factors, listed below, will affect the overall GI of the food.

Fiber

Dietary fiber is the foodstuff that is not digested by the stomach or small intestine. There are two kinds of fiber, insoluble and soluble. Insoluble fibers are not dissolved by water or metabolized by bacteria in the large intestine (examples are cellulose, hemicellulose and lignin). Whole grains are an example of insoluble fiber. Since insoluble fiber is not broken down in the body, it does not yield any calories. By contrast, soluble fiber is dissolved in water and metabolized by the bacteria in the large intestine (examples are pectins, gums and mucilages). Soluble fiber is metabolized into short chain fatty acids and gases, which are absorbed into the blood. Soluble fiber yields about 3 kilocalories/gram.

The type of fiber has an effect on the GI. Finely ground fiber, often found in breads and breakfast cereals, has no or minimal effect on the GI. However, if the fiber is largely intact, it acts as a barrier to digestion. Whole grains containing the entire seed of the plant, which includes the bran, germ and endosperm, have a low GI. Such foods have been suggested to be helpful in weight control since foods high in fiber are “filling” but don't yield much energy. For example, dried beans and peas are a class of vegetable called legumes, which have high fiber content and an enzyme that slows starch digestion. When eaten alone, legumes have a low GI (30-40). Moreover, when eaten with other carbohydrates, they tend to lower the overall GI. Adding beans to rice or pasta, for example, will lower the GI of the meal. Oats and psyllium are other examples of high fiber foods with low GI values. Fiber can also increase the viscosity of the food. More viscous foods are digested more slowly.

Particle Size

The processing of grains (i.e., grinding and milling) reduces particle size. In general, the larger the particle size of food, the lower the GI since less surface area is exposed to digestive enzymes in comparison to refined foods of small particle size. Specific examples of how the particle size of a given food can affect the GI include whole wheat grain vs. cracked wheat, coarse vs. fine flour and rolled vs. milled oats. In each case, the GI is increased by reduction of food particle size. Of note is that fact is that prior to the introduction industrial milling techniques in the nineteenth century, rapidly absorbed starches did not exist in the human diet.

Food Cooking and Processing

Whole foods (i.e., foods in their natural state) will usually have a lower GI than refined and/or processed foods. Processed foods tend to have fiber removed and, therefore, have a higher rate of absorption than unprocessed foods. In addition, the starch in natural foods is stored in compact granules that are relatively hard to digest. Thus, the blanching, boiling and canning of foods significantly affect the GI. In general, cooked starches become more gelatinized, leading to a faster rate of intestinal absorption. For example, uncooked cornstarch may take as long as 8 hours to be digested, absorbed and metabolized to glucose. The potato is another excellent example of this principal. The starch in a potato is stored compactly. If eaten raw, the starch in a potato is very difficult for a human to digest. When baked or boiled, however, a potato is quickly digested (GI approximately 80).

Acidity

The acidity of the food also affects absorption by altering gastric emptying. As a result, more acidic foods are digested more slowly. For example, vinegar and lemon juice will slow carbohydrate absorption. Sourdough bread, which is more acidic than whole wheat or white bread because of the production of lactic and proprionic acid during the yeast fermentation process, is absorbed more slowly than other breads.

Fat

Fat is known to slow carbohydrate absorption. Thus, glucose tablets are recommended for diabetics who are acutely hypoglycemic rather than, for example, a chocolate candy bar that contains a high amount of fat. However, the potential benefits of fat on lowering the GI must be weighed against its increase in caloric intake and long-term health risks. For example, potato chips have a lower GI than a baked potato. While the addition of fat to food may add flavor and lower the GI value, it also significantly increases the caloric density of the food. The benefits of fat on the GI may therefore be outweighed by its disadvantages for most individuals.

General Approach to Carbohydrate-Dosing with Functional Foods

An approach to avoid or minimize the adverse consequences of improper carbohydrate dosing is to modulate serum glucose levels by controlling (“dosing”) carbohydrate intake. Three dosing factors to take into consideration to achieve this goal are, e.g., (1) the food's chemical composition and amount of the food (e.g., the “dose” of the food), (2) the food's physiological effect (i.e., the GI), and the timing of the food ingestion (i.e., the frequency of the dose). To understand how these factors are applied, migraine and headache treatment and prevention is discussed, but these are merely for illustrative purposes, and the same factors can be applied to any disease or condition associated with improper carbohydrate intake. As a general strategy, the present invention describes how to avoid excessive intake of carbohydrate at a single meal while maintaining an adequate supply of liver glycogen, thereby minimizing SNS activation, insulin release, fat storage, and other associated events.

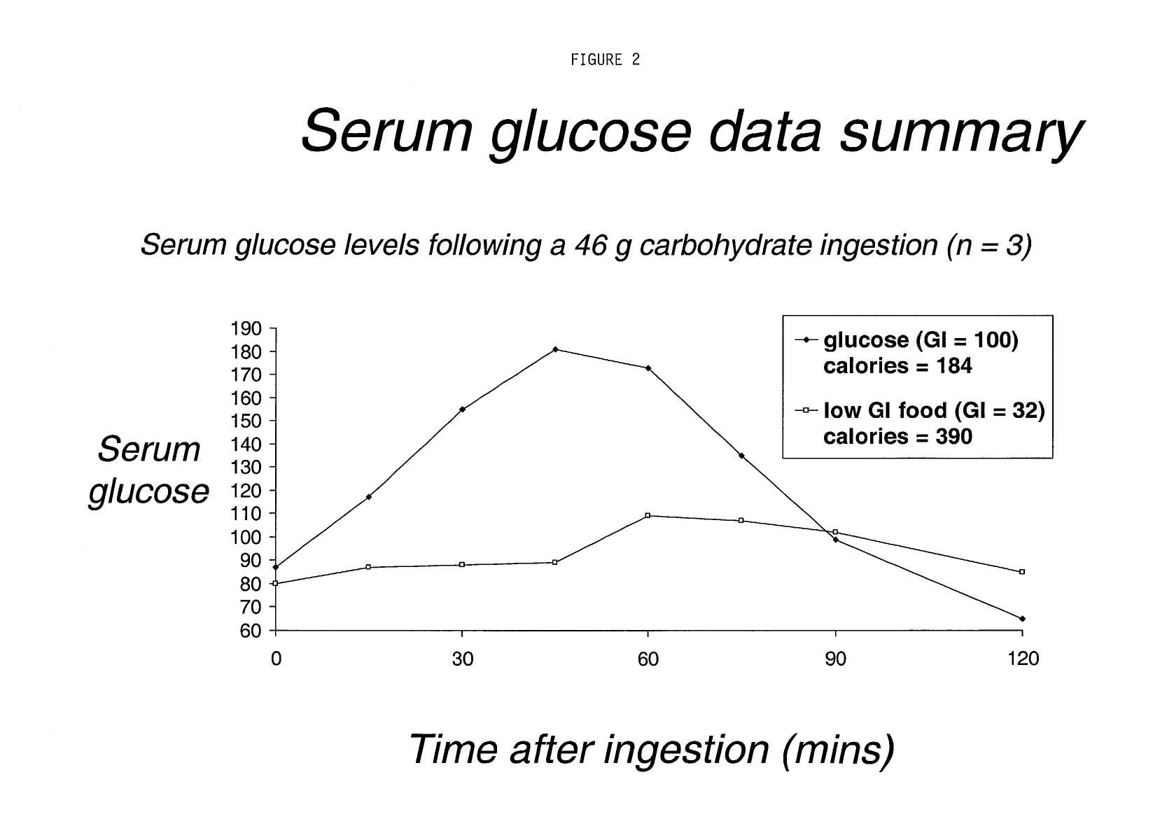

FIG. 2 illustrates an application of these factors. First, the metabolic energy needs of the individual must be determined, a value that can be calculated based on the sex, weight and activity level of an individual. In the example provided, about 15 grams of glucose per hour are required for a given individual. For a three-hour period, this individual would require about 45 grams carbohydrate. If the 45 grams were ingested in the form of glucose, a sharp rise and fall of serum glucose levels would occur, which could precipitate a migraine or headache in a susceptible individual. This adverse effect can be avoided by ingesting a low GI food, e.g., having a GI of about 32. Instead of the peak observed with the high GI glucose, ingestion of the low GI food results in relatively steady serum glucose levels. Since a carbohydrate-containing food having a GI of about one-third of glucose is administered, the insulin and SNS response to the ingested carbohydrate is about one-third that of pure glucose.

One of the adverse affects of improper carbohydrate dosing is activation of the SNS, insulin pathway, gluconeogenesis, etc. For other examples, see, FIG. 1. Accordingly, the present invention relates to compositions and methods for modulating these responses, especially the activation of the sympathetic nervous system, and for treating and preventing disorders and conditions associated with disturbances of these pathways, such as migraine, headaches, stress, and exacerbation of other chronic conditions. In addition, carbohydrate dosing can be used to manage weight.

In accordance with the present invention, an approach to modulating these pathways, such as the SNS, is to control rapid fluctuations in serum glucose levels that lead to migraine, headache, and other disturbances (both short-term and long-term, such as stress) while providing a steady supply of glucose for metabolic needs. When a person is at rest, the brain uses approximately two-thirds of circulating glucose. An average person can store the equivalent of only 80-100 grams of glucose in the liver (the equivalent of about 3 candy bars). However, based on improper carbohydrate dosing, the amount of liver glycogen may be substantially less than the maximal amount, which would make to individual more dependent on gluconeogenesis to supply adequate levels of serum glucose. Meals high in carbohydrates can generate a rapid rise in serum glucose and a secondary increase in insulin secretion from the pancreas. These changes in serum glucose and insulin can lead to adverse physiological consequences, such as prolonged and/or excessive stimulation or activation of the SNS. In certain susceptible individuals, a migraine attack or headache can result or be exacerbated. In addition, low liver glycogen levels make an individual more dependent upon gluconeogenesis, which also leads to adverse physiological consequences, such as prolonged and/or excessive stimulation or activation of the SNS.

The functional foods in accordance with the present invention can contain defined amounts of carbohydrate-containing foods, and other effective components (e.g., fiber and/or fat), for maintaining steady glucose levels that minimize the need to activate the SNS and other physiological consequences of glucose fluctuations. These foods are not only useful in treating and preventing migraine and headaches, but also can be used to mental alertness (e.g., associated with steady glucose levels), control weight, manage pain and other chromic conditions, to treat PMS, etc. As a result, the functional foods are generally useful in the population. They also have special applications to situations where focus, concentration, and endurance are needed (e.g., exam-taking).

In certain susceptible individuals, physiological efforts to maintain serum glucose levels may over-activate or over-stimulate the SNS and other pathways, producing a cascade of adverse events, one of which could be the initiation or exacerbation of a migraine attack, headaches, and other associated disorders. This excessive stimulation of the SNS, for example, triggered by the acute changes in glucose levels or gluconeogenesis can occur in normal individuals, but can also be related to a variety of physiological defects and conditions, including, e.g., an abnormal or hypersensitive SNS, rapid changes in serum glucose, defective insulin pathway, abnormal glucose metabolism, depletion of liver glycogen stores, etc.

Intervention in, augmentation of, or inhibition of, etc., any of the physiological processes that modulate serum glucose concentrations can be utilized to treat and prevent the conditions mentioned herein, such as to prevent or treat headaches and migraine attacks, and other associated symptoms. This includes, e.g., administering effective functional foods and pharmacological agents, and applying therapies, such as dietary regimes, that modulate glucose levels, either directly or via secondary means. As discussed in more detail below, the present invention involves the concepts of administering calculated dosages of functional foods to prevent and ameliorate disturbances produced in the physiological pathways related to carbohydrate intake, such as migraine and headache, as well as to “fuel” the energy needs of the brain and body with functional foods. A migraine attack or headache can be treated and/or prevented in such susceptible individuals by ingesting a food which prevents, reduces, diminishes, etc., the need to activate the sympathetic nervous system in the maintenance of serum glucose levels. Any method, agent, composition, food, dietary regime, etc., which prevents, decreases, diminishes, etc., rapid fluctuations in serum glucose, while providing a steady supply of glucose for metabolic needs, can be used to treat and prevent migraine and headache attacks.

Managing Ingestion of Functional Foods

An approach in accordance with the present invention to dose a subject with effective amounts of carbohydrate-containing functional foods is to administer (e.g., ingest orally) a food at regular intervals that results in the relatively slow release of carbohydrate into the serum, thereby reducing the risk of rapid changes in serum glucose levels or the need for gluconeogenesis, and at the same time avoiding fat storage which leads to weight gain. For instance, the glucose/insulin/SNS response can be modulated by minimizing, or eliminating, the intake of glucose and other high GI carbohydrates. Serum glucose fluctuations after meals that are rich in high GI carbohydrates are steep, and result in a relatively significant activation of the SNS compared to meals that produce smaller fluctuations in serum glucose. “Fasting” to the point of liver glycogen depletion also activates the SNS.

To prevent such over-activation or prolonged stimulation of the SNS, meals can be eaten at regular intervals and can contain low GI carbohydrates or higher GI carbohydrates accompanied by components which slow absorption such as fiber and/or fat. Thus, the consumption of natural fiber, fat, and/or low GI carbohydrates can prolong the absorption of a given carbohydrate caloric load and thereby reduce the serum glucose and insulin fluctuations.

A functional food refers to an effective food in accordance with the present invention that is useful for achieving any of the mentioned goals. One approach is to administer low GI foods that, after ingestion, produce less rapid changes in serum glucose than are observed after a high GI carbohydrate, such as glucose, is ingested, despite having the same caloric and carbohydrate content as a high GI food. For instance, an effective food in accordance with the present invention comprises one or more agents that slow down the absorption and/or digestion of carbohydrates. These agents, include, but are not limited, to low GI carbohydrates (e.g., fructose, whole grain rice, pasta), fiber, fat and any other agent which prolongs, depresses, inhibits, etc., carbohydrate absorption from the GI tract or glucose metabolism. Fiber refers to a material, generally from plants, which is resistant to digestion by the human small intestine. Useful low GI carbohydrates, include, but are not limited to, fructose, certain amylose starches and non-starch polysaccharides. Fructose is the preferred sugar to maintain liver glycogen stores. Useful fibers include, but are not limited to, e.g., guar, pectin, gum tragacanth, methylcellulose, wheat brain, cellulose, etc.

Table 1 provides examples of some foods and their GI values. As illustrated in the Table 1, none of the above products meet all of the ideal requirements. Pretzels have a high GI (83) and are very high in complex carbohydrates but low in fat. By contrast, Peanut M&Ms have a low GI but are very high in fat and calories. According to the current invention, the preferred current product in the above list would be the yogurt preparations, which have a relatively low GI (33) and are relatively low in fat and calories. A preferred functional food product could have a low GI (<50 and preferably <30), relatively low in calories, possibly containing some fat to decrease absorption, and some fiber. The present invention relates to such preferred functional foods.

Food and associated agents can be administered at any times, and in any amounts, that are effective for controlling serum glucose levels, e.g., to prevent or treat a migraine attack or headache, to control weight, to control stress, etc. In general, a goal is to administer amounts of foods that minimize the fluctuation of serum glucose levels while providing an adequate caloric intake, especially in terms of carbohydrate needs. The frequency and amount of food ordinarily depends on various factors, including patient weight, height, basal metabolic rate, health, gender, activity level (e.g., how much exercise per day), resting level (e.g., how much sleep per night), and other individual differences (such as the defect responsible for migraine susceptibility).

In an example of a useful therapy, an individual could ingest 3 “normal” meals with the addition, for example, of 3 dietary supplements in the mid-morning (e.g., 2-3 hours after breakfast and 2-3 hours before lunch), mid-afternoon (e.g., 2-3 hours after lunch and 2-3 hours before dinner), and at bedtime (e.g., 2-4 hours after dinner). The caloric value of each of these food supplements for an average adult can be about in the 80-180 calorie range, where each supplement comprises about greater than about 50% of the calories from low GI carbohydrates, preferably greater than about 60% or 75% low GI carbohydrates. The number and frequency of dietary supplements can be adjusted to suit the particular individual (e.g., an additional supplement after dinner, but before bedtime). The caloric content and amounts of low GI carbohydrate can be adjusted, depending on the fiber or fat content (e.g., low GI carbohydrate percentage can be decreased if fiber and/or fat is added), and the weight of the individual.

A specifically designed functional food can be ingested at the time of awakening if the individual does not plan on eating breakfast within the next hour or so, and every about 3-4 hours thereafter until a regular meal is ingested. Similarly, a specifically designed food can be ingested about 3-4 hours after lunch if another meal is not planned within then next hour or so (a typical situation). A bedtime food ingestion is also recommended if the individual has not eaten for the past about 3-4 hours and suffers from early morning headache.

The dietary regimes disclosed in Dexter et al., Headache, 18:91-94, 1978, and Wilkinson, Am. J. Med. Sci. 218:209-212, 1949 (which are hereby incorporated by reference in their entirety), are expressly excluded from the present invention.

An example of a suggested therapy to attenuate or stabilize a migraine attack or headache in a susceptible individual can be the use of the food supplement during an actual episode. Although there are a very wide variety of trigger factors (e.g., stress, weather changes, hormonal fluctuations, etc.) that may initiate and maintain a migraine attack, maintenance of stable and adequate glucose levels during an attack may lead to more rapid resolution of the attack. Therefore, avoidance of high GI carbohydrates during a migraine attack and ingestion of low GI food supplement with adequate carbohydrates (described above) at 3-4 hour intervals is recommended.

Methods and Compositions

The present invention relates to methods for treating or preventing one or more symptoms of a migraine attack, headache, and other related disturbances, managing weight, controlling stress, enhancing memory, enhancing concentration, treating PMS, depression and other neuropsychiatric disorders, and chronic pain, comprising, e.g., administering, to a subject in need thereof, an amount of a carbohydrate-containing food which is effective in minimizing activation of the sympathetic nervous system, minimizing release of insulin, etc. for a predetermined time, and which provides an adequate glucose supply to the central nervous system. The food can be additionally effective to, e.g., maintain substantially uniform blood glucose levels over said predetermined time, dampen blood serum glucose fluctuations, maintain serum glucose at a level which minimizes stimulation of the sympathetic nervous system, minimize gluconeogenesis, minimize insulin release, minimize release of epinephrine, norepinephrine, or cortisol.

By the phrase “effective in minimizing activation of the sympathetic nervous system,” it is meant that, when the carbohydrate-containing food is administered (i.e., ingested by the subject), the resulting change in serum glucose levels are insufficient to produce the quantity of SNS stimulation or activation that evokes or maintains a migraine attack or headache, or to produce any of the adverse consequences of excessive carbohydrate intake described herein. As described elsewhere, SNS activation refers to the physiological changes in the SNS that occur when it is in operation, e.g., responding to levels of blood glucose, releasing neurotransmitters, such as epinephrine and norepinephrine, secreting hormones, etc.

It is recognized that some stimulation can be tolerable, but excessive or prolonged stimulation (“over-activation” or “over-stimulation”), especially in susceptible individuals, can result in the initiation, prolongation or increase in severity of a migraine or other headache attack, and other pathological conditions. In other words, e.g., a food which is ineffective in minimizing SNS stimulation does not prevent or treat a migraine or headache, but instead, can induce an attack, or can create physiological conditions that make an attack more likely.

The carbohydrate-containing food minimizes SNS activation for “a predetermined time.” This indicates that the amount of carbohydrate ingested by the subject can be calculated to minimize the SNS activation for a specific period of time, e.g., 2 hours, 4 hours, 8 hours (e.g., when it is administered prior to bedtime), etc. The carbohydrate-containing food can therefore be viewed analogously to a drug, where a specific dosage is administered to a subject to achieve a pharmacological effect over a specific period of time.

A “carbohydrate-containing food,” or simply “food” or “food source,” can be any composition which is normally ingested as an energy source and which contains carbohydrate, including foods that have fiber and other non-digestible components. The term “functional food” can be used interchangeably. As already discussed above, rapid changes in serum glucose levels can result in the over-activation of the SNS in certain susceptible individuals. By administering effective amounts of foods and other agents, activation of the SNS can be modulated, i.e., preventing, reducing, delaying, suppressing, minimizing, lowering, diminishing, decreasing, increasing, maximizing, SNS activation or stimulation. In preferred embodiments of the invention the modulatory effect is aimed at the preventing, reducing, etc., SNS activation via dietary modulation. If dietary activation of the SNS is avoided, or at least reduced, symptoms produced or exacerbated by SNS activation can be avoided, stabilized or improved (e.g., migraine attacks or headaches).

The amount of carbohydrate-containing food that is administered also provides an adequate glucose supply to the brain (and other glucose-dependent body structures or tissues, such as red blood cells and the kidneys). By this, it is meant that sufficient glucose is provided to the brain and body to meet its energy needs for carbohydrates. For example, the brain of an average adult person requires about 4-5 grams of glucose per hour for basic energy needs. The energy needs of the remainder of the body can be met by about 2-4 grams of glucose per hour. Thus, a supply of glucose (or equivalents thereof) of about 6-9 grams per hour would be necessary to ensure that the baseline metabolic energy needs of the brain and body for glucose were met. However, the minimal amount of glucose needed per hour is dependent upon the weight of the individual, the physical activity level of the individual, of the amount of neuronal activity and other physiological factors. For instance, fructose is a useful carbohydrate-containing component to maintain serum glucose levels during long periods, such as sleep, when feeding is constrained. As a general rule, a constant supply of 10-20 g of glucose per hour should be an adequate supply of glucose for most individuals during waking hours.

An effective food source can also be used to maintain a substantially uniform blood glucose level. During the post-absorptive state, homeostatic glucose levels can be about, e.g., 80-100 mg glucose/100 ml. Assuming that this is the baseline, any upward or downward deviations can result in activation of the SNS, migraine, and other pathological conditions and disorders. A “substantially uniform” glucose level means a level that is insufficient to produce or provoke such effects. Such levels can be, e.g., about 10%-30%, or less, of normal homeostatic amounts. It is expected that there will be differences between individuals in the amount of glucose fluctuation that can be tolerated, without over-activation or over-stimulation of the SNS.

Carbohydrate-containing food sources and other agents can also be administered to dampen blood serum glucose and insulin fluctuations. By the phrase, “dampen,” it is meant that fluctuations in serum glucose and insulin are flattened in such a manner as to prevent or reduce SNS over-activation. High GI foods, such as glucose, after ingestion of 50 g, can produce in susceptible individuals pronounced changes in serum glucose levels, e.g., changing from 80-100 mg. glucose/100 ml to as high 170 mg glucose/100 ml in thirty to sixty minutes or less. On the other hand, the same caloric load of a low GI carbohydrate food, or a high GI carbohydrate in combination with fat, fiber, or other agent which delays or slows down absorption, in comparison with a high GI carbohydrate food alone, will produce both a lower maximal serum glucose concentration as well as a slight delay in the time of the peak serum level.

To maintain adequate levels of glucose after prolonged fasting or strenuous exercise, the body may initiate gluconeogenesis. This process uses three and four carbon precursors (generally non-carbohydrates), such as amino acids, TCA intermediate, and lactate, and converts them into glucose. The liver is the primary gluconeogenic organ in the body, along with the kidney cortex. Gluconeogenesis requires activation of the SNS. An amount of a carbohydrate-containing food can be administered, e.g., prior to a fast or strenuous exercise, that is sufficient to minimize SNS activation of the sympathetic nervous system. For instance, if a susceptible subject is going to sleep for 6-8 hours, or engage in strenuous activity, a carbohydrate-containing food can be ingested that avoids SNS activation produced by prolonged fasting. Fructose is the preferred carbohydrate in this example since it will be stored in the liver as glycogen until needed.

In addition to the aforementioned activities, an effective food can also modulate the release of hormones, neurotransmitters, and other messengers that regulate glucose metabolism and therefore impact on the SNS. For instance, the release of norepinephrine by the SNS in response to rapid changes in glucose is one of the earliest events that occurs during post-absorptive metabolism. This release can initiate a cascade of events that culminate in a headache or migraine attack in susceptible individuals, or any other of the mentioned conditions. By minimizing the release of norepinephrine, these conditions can prevented.

Other chemical messengers, in addition to norepinephrine, play a role in glucose metabolism, and can initiate or exacerbate conditions such as headaches, migraine attacks, and stress. These include, but are not limited to, insulin, glucagon, epinephrine, growth hormone, and cortisol. The latter three can stimulate gluconeogenesis and lipolysis during a fasting period, maintaining serum glucose levels via SNS stimulation Avoiding or “minimizing” their release, can therefore be preventative or therapeutic.

An aspect of the present invention is the administration of a carbohydrate-containing food that has been selected on the basis of its glycemic index (e.g., “where the glycemic index has been used as selection factor to select a carbohydrate-containing food”). This indicates an active “selecting step” in which a food is chosen for its glycemic index and the result that is to be accomplished, e.g., minimizing activation of the sympathetic nervous system for a predetermined time, providing an adequate glucose supply to the central nervous system, maintaining substantially uniform blood glucose levels, dampening blood serum glucose fluctuations, maintaining serum glucose at a level which minimizes stimulation of the sympathetic nervous system, minimizing gluconeogenesis, minimizing insulin release, and/or minimizing release of epinephrine, norepinephrine, or cortisol. In other words, the glycemic index is used as a “selection factor” to chose which functional food, and how much of it, is to be administered. Other factors can also be taken into consideration, including, but not limited to, taste, texture, color, the presence of natural and/or organic ingredients, sugar content, fat content, fiber content, protein content, calories, weight of an effective dose, time period over which the desired effect is to last (“predetermined time”), etc. Glycemic index is discussed in more detail below.

The present invention also relates to methods of formulating a functional food to provide an adequate supplies of glucose, over predetermined times, for treating or preventing migraine attack or headache, for providing an adequate supply of glucose to the brain, for weight management, for controlling stress, for treating and/or preventing chronic or recurring conditions, such as pain and PMS, comprising, e.g., one or more of the following steps in any effective order: selecting doses of food using parameters for brain glucose utilization, said parameters comprising: at least one food with a known glycemic index, rate of brain glucose utilization, and predetermined time period; and selecting doses of food components using parameters for body glucose utilization, said parameters comprising: at least one food with a known glycemic index, rate of body glucose utilization, and predetermined time period, wherein said functional food is effective in minimizing action of the sympathetic nervous system.

A “functional food” is a food which, when administered, achieves a desired result, i.e., the food has a function to perform. It can mean any composition that is normally ingested as an energy source, including foods that comprise non-digestible entities, such as fiber. In accordance with the present invention, functional foods can be utilized to treat and/or prevent migraine, control and manage body weight, reduce stress, enhance memory, enhance concentration, treat and/or prevent premenstrual syndrome (PMS), treat and/or prevent depression, chronic pain and other neuropsychiatric disorders, minimize SNS activation, minimize the insulin response, and, provide the brain with an adequate supply of glucose, etc. These functional foods can be designed using (“formulated”) using various selection factors, depending upon the specific results to be achieved. For example, a functional food can be designed based on the brain's glucose utilization requirements and/or the body's glucose utilization requirements. By the term “body,” it is meant all organs and tissues of the mammalian body with the exception of the brain.

Parameters that can be used to formulate a functional food include, e.g., a food's glycemic index, rate of brain and/or body glucose utilization, and predetermined time period over which the functional food is effective. To illustrate this, consider the following example where a mid-day snack is formulated whose goal is to minimize SNS activation over a three-hour time period. In this case, the functional food can be administered at about 3 pm in order to satisfy food craving without having adverse effects on the SNS, as well as minimizing insulin release. Assume that the brain and body of an average person utilizes a minimum of about 10 grams glucose per hour (“rate of brain glucose utilization”). The functional food's effect is to last about 3 hours (“predetermined time”). This means that the functional food should provide 30 grams of glucose.

In order to minimize SNS activation and insulin release, avoid gluconeogenesis, and yet maintain steady glucose levels, a proportional amount of a low GI carbohydrate-containing food can be selected. For instance, if a food having a GI of about 33 (e.g., yogurt) is administered at a “dose” of 30 g, then the SNS activation, insulin release, etc. would be only about 33% of the SNS activation, insulin release, etc. that would occur if 30 g glucose (or any other food with a GI=100) were ingested. The entire “dose” of 30 g carbohydrate would thus be metabolically equivalent to a single dose of 10 g glucose (or any other food with a GI=100) in terms of SNS activation, insulin release, etc. This “dosing” regimen would achieve the desired goal of providing a steady and adequate supply of glucose the brain and other dependent body parts with a significantly lower amount of physiological stress than a 30 g dose of glucose.

Additional selection factors can be utilized to design or formulate functional foods in accordance with the present invention. For instance, as mentioned, a “functional food” can comprise, e.g., about 55%, 60%, 65%, 70%, 75%, 80% or more, of its total calories, of a low GI carbohydrate, with the remainder of the calories possibly coming from fat and/or protein. High GI carbohydrates, such as glucose, may be desired to enhance the flavor, taste, and palatability of a “functional foods”. Thus, a “functional food” does not have to be high GI carbohydrate restricted. When a “functional food” contains a high GI carbohydrate, fiber and/or fat can be included to slow its release into the blood. A “functional food” can comprise, e.g., 10%, 20%, 30%, etc., of its calories from high GI carbohydrates, which are balanced by the presence of low GI carbohydrates, fats, and/or fiber.

Functional foods comprising a carbohydrate containing food components for providing the brain with an adequate supply of glucose, for treating or preventing migraine attack or headache, etc., can comprise, e.g., an effective dose of a carbohydrate containing food component having a preselected glycemic index and amount for delivering an amount of glucose to the brain while minimizing activation of the sympathetic nervous system and/or maintaining a substantially uniform blood glucose levels, and, an effective amount of a carbohydrate containing food component having a preselected glycemic index and amount for delivering an amount of glucose to other glucose-dependent tissues (e.g., red blood cells, kidney, and other body tissues) while minimizing activation of the sympathetic nervous system and/or maintaining a substantially uniform blood glucose levels. The term “dose” is used to indicate the pharmacological precision with which functional foods are formulated and administered. As discussed in detail above, these amounts can be selected to achieve precise goals, e.g., minimize SNS activation, minimize gluconeogenesis, maintain steady glucose levels, to treat and/or prevent migraine attacks and headaches, etc. Glucose is a drug that has profound physiological effects on the brain and body, and therefore functional foods are, in effect, pharmacological agents that are carefully formulated by dosage to produce targeted and expected effects on the subject's physiology.

The present invention also relates to methods for treating or preventing migraine attack or headache, and/or providing an adequate supply of glucose to the brain, and/or for weight management, controlling stress, treating and/or preventing chronic or recurring conditions, such as pain and PMS, comprising, e.g., prescribing to a subject in need thereof a low glycemic food which is effective to minimize blood glucose fluctuations, wherein said food has been selected using its glycemic index as a selection factor. A health care professional, such as a physician, nurse, physician's assistant, dietician, pharmacist, or acupuncturist, may direct a patient to use a low glycemic food to treat or prevent a headache or migraine attack. The direction need not be in writing, but may be oral. Thus, the term “prescribing” is used in the general sense to mean a recommendation or direction by a health care professional to consume a food in accordance with the present invention and does not require that a classical “prescription” type order is written or recorded with a pharmacist.

Functional foods of the present invention can be distributed to patients in need thereof through a variety of different routes, including by prescribing (e.g., as described above), advertising (e.g., in print media, television, internet, radio, cable, movies, email, mail, etc.), word of mouth, etc. One aspect of the present invention relates to advertising a functional food for treating or preventing migraine attack or headache, and/or providing an adequate supply of glucose to the brain, and/or for weight management, controlling stress, treating and/or preventing chronic or recurring conditions, such as pain and PMS, in a subject in need thereof, wherein said food has been selected using its glycemic index as a selection factor. Advertising is used in the general sense to mean to announce or praise the product in a medium of public communication in order to induce people to buy or use it.

Managing Weight

As already discussed, the present invention also relates to methods and compositions for managing weight. In addition to the guidance set forth above, some further considerations for carbohydrate dosing when used in the weight management context include, e.g., calculating the required dose of carbohydrate, not exceeding about 40-60 grams of carbohydrate per meal for an average individual, eating foods with a low GI, and eating isocaloric foods with the highest serving weight as possible. For instance, shortly after wakening, a breakfast can be eaten which contains about a 4-5 hour dose of carbohydrate. Snacks can be used as “bridges” to provide about a 2-4 hour dose of carbohydrate between meals. Bedtime snacks can be administered to provide a steady nighttime supply of glucose and to replenish or maintain liver glycogen stores, thereby avoiding nocturnal gluconeogenesis.

Pharmaceutical Agents

Pharmaceutical agents can also be used alone, or in combination with “functional foods”, to treat migraine in accordance with the present invention. As mentioned, glucose can rise exponentially after a meal rich in high GI carbohydrates, producing a sharp drop in glucose levels secondary to induced insulin release that, again, results in SNS activation. For instance, a rise in serum glucose concentrations followed by meal ingestion stimulates the release of insulin from the beta cells of the pancreas. Insulin, in turn, promotes the increased utilization of glucose, e.g., by stimulating glucose uptake into cells and by inhibiting glycogenesis. Any agent which interferes or diminishes insulin's effects on glucose levels can be used in accordance with the present invention, e.g., by dampening the fall in serum glucose. Such agents, include, e.g., insulin antagonists, such as glucagon, alpha-2-adrenergic agonists, beta-2-adrenergic antagonists, and derivatives and analogs thereof. For other examples, see, e.g., Davis and Granner, Goodman and Gilman's Pharmacological Basis of Therapeutics, Chapter 60, Ninth Edition.

On the hand, pharmaceutical agents can also be utilized which enhance insulin's effects, reducing the initial sharp rise in glucose levels. Such agents can be referred to as “hypoglycemic agents,” and include, e.g., alpha-2-adrenergic antagonists, beta-2-adrenergic agonists, gastrin, secretin, CCK, VIP, gastrin-releasing peptide, enteroglucagon, insulin, sulfonylureas, gliclazide, glipizide, acetohexamide, tolazamide, chlorpropamide, tolbutamide, metformin, ciglitazone, pioglitazone, alpha-glucosidase inhibitors, and derivatives and analogs thereof. For other examples, see, e.g., Davis and Granner, Goodman and Gilman's Pharmacological Basis of Therapeutics, Chapter 60, Ninth Edition. These and other drugs can be administered conventionally, e.g., as used in the treatment of diabetes.

Agents can also be utilized in conjunction with functional foods, which modulate liver glycogen, including agents which modulate gluconeogenesis.

Foods, etc., can also be used in combination with standard treatments for migraine, including such agents as aspirin, acetaminophen, ibuprofen, indomethacin, naproxen, isometheptene, acetaminophen, dichloralphenazone, ergotamine, dihydroergotamine, sumatriptan, ergot alkaloids, calcium channel modulators, etc. For other examples, see, e.g., Peroutka, S. J., Goodman and Gilman's Pharmacological Basis of Therapeutics, Chapter 21, Ninth Edition.

Subjects

Any subject can be treated in accordance with the present invention, including, but not limited to, subjects who experience migraines and/or headaches, stress, weight management difficulty, chronic or recurring conditions, such as pain and PMS, and SNS over-stimulation. A “migraine attack” includes any symptoms associated with migraine, including, e.g., headache, throbbing, nausea, vomiting, photophobia, sonophobia, etc. as is generally accepted by the worldwide medical community. Any migraine type can be treated in accordance with the invention, including, e.g., migraine without aura, migraine with aura, migraine with typical aura, migraine with prolonged aura, familial hemiplegic migraine, basilar migraine, migraine aura without headache, migraine with acute-onset aura, ophthalmoplegic migraine, retinal migraine, etc.

Headaches can also be treated or prevented in accordance with the present invention. These include, but are not limited to, tension headaches (e.g., pressure and aching), muscle contraction headaches, non-throbbing or steady headache, etc, as commonly defined by the medical community. The term tension-type headache is commonly used to describe a chronic head pain syndrome characterized by bilateral tight bandlike discomfort. Patients may report that the head feels as if it is in a vise or that the posterior neck muscles are tight. The pain typically builds slowly, fluctuates in severity, and may persist more or less continuously for many days.

Any person who experiences SNS disturbances, headaches, migraines, etc., can benefit from a “functional foods” in accordance with the present invention. Persons who exhibit a normal, hypoglycemic, or other abnormal serum glucose response after a standard glucose tolerance test can utilize the foods and methods described herein to prevent or treat SNS disturbances, and both short-term and long-term conditions associated therewith.

| | Serving | | Total | Total | Dietary | | | Glycemic |
|---|---|---|---|---|---|---|---|---|
| Food and snacks | Size | Calories | Fat | Carb | Fiber | Sugars | Protein | Index |
| Pure Protein Bar | 50 | 180 | 2.5 | 9 | 0 | 3 | 20 | 22 |
| (Peanut Butter) | | | | | | | | |
| Cherries (20 cherries) | 136 | 100 | 1 | 22 | | | 1 | 22 |
| Peanut M&Ms (18) | 46 | 240 | 12 | 28 | 2 | 23 | 4 | 32 |
| Yogurt with fruit | 227 | 210 | 2 | 40 | <1 | 38 | 9 | 33 |
| (Dannon) | | | | | | | | |
| Oatmeal (½ cup dry) | 40 | 150 | 3 | 27 | 4 | 1 | 5 | 49 |
| Oatmeal (¾ cup dry) | 60 | 225 | 5 | 40 | 6 | 1 | 7 | 49 |
| [usual meal] | | | | | | | | |
| Potato chips (Ruffles) | 28 | 160 | 10 | 14 | 1 | 0 | 2 | 54 |
| (12 chips) | | | | | | | | |
| Banana (one medium) | 114 | 105 | 1 | 27 | | | 1 | 55 |
| Popcorn (½ bag) | 50 | 240 | 15 | 27 | 5 | | 5 | 55 |
| Kellogg's Sugar | 31 | 120 | 0 | 28 | 1 | 12 | 1 | 55 |
| Frosted Flakes (¾ | | | | | | | | |
| cup; 1.1 oz/half-bowl) | | | | | | | | |
| [½ usual meal] | | | | | | | | |
| PowerBar | 65 | 230 | 2 | 45 | 3 | 14 | 10 | 57 |
| Pizza (one slice) | 126 | 300 | 12 | 38 | 3 | 3 | 11 | 60 |
| Raisins (¼ cup) | 40 | 130 | 0 | 31 | 2 | 29 | 1 | 64 |
| Glucose | 40 | 160 | 0 | 40 | 0 | 40 | 0 | 100 |

### EXAMPLES

### Example 1

### Prototype Bar Composition

Carbohydrate (40 g Total)

No more than 10 g glucose or other high GI carbohydrate

fructose preferred over glucose

amylose preferred over amylopectin

uncooked cornstarch should be considered

Fat (2.5 g or Less)

None preferred

Protein

None preferred

Fiber (3 g)

oat soluble fiber preferred

Serving Weight

Maximize

Serving Size

Maximize

Other Contents

May also contain as much as 20% minimal daily requirement of Vitamin A, C, calcium, iron, etc.

May also include potential migraine-related constituents such as riboflavin, magnesium, iron, calcium, vitamin B6, Vitamin E, etc

Other

The topic headings set forth above are meant as guidance where certain information can be found in the application, but are not intended to be the only source in the application where information on such topic can be found.

The preceding description, utilize the present invention to its fullest extent. The preceding preferred specific embodiments are, therefore, to be construed as merely illustrative, and not limiting the remainder of the disclosure in any way whatsoever. The entire disclosure of all applications, patents and publications, cited above and in the figures are hereby incorporated by reference in their entirety.

1. A method of treating or preventing one or more symptoms of a migraine attack or headache in a subject in need thereof, comprising:
administering an amount of a carbohydrate-containing food which is effective in minimizing activation of the sympathetic nervous system for a predetermined time, and which provides an adequate glucose supply to the central nervous system and other glucose dependent tissues.

2. A method of claim 1, wherein said food is effective to maintain substantially uniform blood glucose levels over said predetermined time.

3. A method of claim 1, wherein said food is effective to dampen blood serum glucose fluctuations.

4. A method of claim 1, wherein said food is effective to maintain serum glucose at a level which minimizes stimulation of the sympathetic nervous system.

5. A method of claim 1, wherein said food is effective to minimize or prevent gluconeogenesis.

6. A method of claim 1, wherein said food is effective to minimize insulin release.

7. A method of claim 1, wherein said food is effective to minimize release of epinephrine, norepinephrine, or cortisol.

8. A method of claim 1, wherein said food is administered every 2-4 hours after a regular meal.

9. A method of claim 1, wherein said food is administered prior to bedtime.

10. A method of claim 9, where said food is effective to replenish or maintain liver glycogen stores, thereby avoiding nocturnal gluconeogenesis.

11. A method of claim 1, wherein said food was selected on the basis of its glycemic index.

12. A method of treating or preventing one or more symptoms of a migraine attack or headache in a subject, comprising:
administering to a subject in need thereof an effective amount of a selected food which provides an adequate glucose supply to the central nervous system and other glucose dependent tissues for a predetermined time, wherein the glycemic index has been used as selection factor to select said food.

13. A method of claim 12, wherein said selected food is effective in minimizing activation of the sympathetic nervous system for a predetermined time.

14. A method of treating or preventing a migraine attack or headache in a subject, comprising:
selecting a food which is effective to minimize activation of the sympathetic nervous system for a predetermined amount of time using the food's glycemic index as a selection factor, and
administering the selected food to a subject in need thereof.

15. A method of formulating a functional food to provide an adequate supply of glucose for treating or preventing migraine attack or headache, comprising:
selecting doses of food using parameters for brain glucose utilization, said parameters comprising:
at least one food with a known glycemic index,
rate of brain glucose utilization, and
predetermined time period,
selecting doses of food components using parameters for body glucose utilization, said parameters comprising:
at least one food with a known glycemic index,
rate of body glucose utilization, and
predetermined time period,
wherein said functional food is effective in minimizing action of the sympathetic nervous system.

16. A functional food for supplying an adequate supply of glucose to the brain for a predetermined amount of time, or, for treating or preventing migraine attack or headache, said food comprising
an effective amount of a function food produced by a method of claim 15.

17. A functional food comprising food components for providing the brain with an adequate supply of glucose or for treating or preventing migraine attack or headache, comprising:
an effective dose of a carbohydrate containing food component having a preselected glycemic index and amount for delivering an amount of glucose to the brain while minimizing activation of the sympathetic nervous system, and
an effective of a carbohydrate containing food component having a preselected glycemic index and amount for delivering an amount of glucose to other glucose-dependent tissues while minimizing activation of the sympathetic nervous system.

18. A functional food of claim 17, further comprising food components comprising fat and/or fiber.

19. A method of treating or preventing a migraine attack or headache in a subject,
comprising:
prescribing to a subject in need thereof a low glycemic food which is effective to minimize blood glucose fluctuations, wherein said food has been selected using its glycemic index as a selection factor.

20. A method of selling a functional food for treating or preventing a migraine attack or headache in a subject, comprising:
advertising a functional food for treating or preventing a migraine attack or headache in a subject in need thereof, wherein said food has been selected using its glycemic index as a selection factor.

21. A method of providing to a subject, in need thereof, an adequate supply of glucose to the brain for a predetermined period of time, comprising:
administering an effective dose of a functional food comprising carbohydrate and other components which deliver a predetermined amount of glucose for a predetermined amount of time and which provide an adequate supply of glucose to the brain, wherein said functional food has been selected by its glycemic index and is effective for minimizing activation of the sympathetic nervous system.

22. A method of claim of claim 21, wherein food is administered prior to an exam or prior to going to sleep.
